(19) United States
(12) Patent Application Publication
(10) Pub. No.: US 2024/0087686 A1
(43) Pub. Date: Mar. 14, 2024

(54) PREDICTING COMPLETE PROTEIN REPRESENTATIONS FROM MASKED PROTEIN REPRESENTATIONS

(71) Applicant: DeepMind Technologies Limited, London (GB)

(72) Inventors: Alexander Pritzel, London (GB); Catalin-Dumitru Ionescu, London (GB); Simon Kohl, London (GB)

(21) Appl. No.: 18/273,594

(22) PCT Filed: Jan. 27, 2022
(86) PCT No.: PCT/EP2022/051943

§ 371 Date: Jul. 21, 2023

Related U.S. Application Data
(60) Provisional application No. 63/161,789, filed on Mar. 16, 2021.

(51) Int. Cl. G16B 40/20; G16B 15/20; G16B 15/30; G16B 20/50; G16B 30/00

(52) U.S. Cl. CPC G16B 40/20; G16B 15/20; G16B 15/30; G16B 20/50; G16B 30/00

(57) ABSTRACT

Methods, systems, and apparatus, including computer programs encoded on a computer storage medium, for unmasking a masked representation of a protein using a protein reconstruction neural network. In one aspect, a method comprises: receiving the masked representation of the protein; and processing the masked representation of the protein using the protein reconstruction neural network to generate a respective predicted embedding corresponding to one or more masked embeddings that are included in the masked representation of the protein, wherein a predicted embedding corresponding to a masked embedding in a representation of the amino acid sequence of the protein defines a prediction for an identity of an amino acid at a corresponding position in the amino acid sequence, wherein a predicted embedding corresponding to a masked embedding in a representation of the structure of the protein defines a prediction for a corresponding structural feature of the protein.

### BACKGROUND

This specification relates to predicting complete protein representations from masked protein representations.

A protein is specified by one or more sequences of amino acids. An amino acid is an organic compound which includes an amino functional group and a carboxyl functional group, as well as a side-chain (i.e., group of atoms) that is specific to the amino acid.

Protein folding refers to a physical process by which a sequence of amino acids folds into a three-dimensional configuration. The structure of a protein defines the three-dimensional configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein reconstruction system implemented as computer programs on one or more computers in one or more locations that can unmask a masked representation of a protein using a protein reconstruction neural network. The protein reconstruction neural network is not limited to having a particular architecture, and as described later the system can improve the accuracy of a protein representation by jointly processing representations of both an amino acid sequence and a structure of the protein.

As used throughout this specification, the term “protein” may be understood to refer to any biological molecule that is specified by one or more sequences of amino acids. For example, the term protein may be understood to refer to a protein domain (i.e., a portion of an amino acid sequence that can undergo protein folding nearly independently of the rest of the amino acid sequence) or a protein complex (i.e., that is specified by multiple associated amino acid sequences).

Throughout this specification, an embedding refers to an ordered collection of numerical values, e.g., a vector or matrix of numerical values.

According to a first aspect, there is provided a method performed by one or more data processing apparatus for unmasking a masked representation of a protein using a protein reconstruction neural network, the method comprising: receiving the masked representation of the protein, wherein the masked representation of the protein comprises: (i) a representation of an amino acid sequence of the protein that comprises a plurality of embeddings that each correspond to a respective position in the amino sequence of the protein, and (ii) a representation of a structure of the protein that comprises a plurality of embeddings that each correspond to a respective structural feature of the protein, wherein at least one of the embeddings included in the masked representation of the protein is masked; and processing the masked representation of the protein using the protein reconstruction neural network to generate a respective predicted embedding corresponding to one or more masked embeddings that are included in the masked representation of the protein, wherein a predicted embedding corresponding to a masked embedding in the representation of the amino acid sequence of the protein defines a prediction for an identity of an amino acid at a corresponding position in the amino acid sequence, wherein a predicted embedding corresponding to a masked embedding in the representation of the structure of the protein defines a prediction for a corresponding structural feature of the protein.

In some implementations, the method further comprises: updating the masked representation of the protein by replacing a proper subset of the masked embeddings in the masked representation of the protein by corresponding predicted embeddings; and processing the updated masked representation of the protein using the protein reconstruction neural network to generate respective predicted embeddings corresponding to one or more remaining masked embeddings that are included in the masked representation of the protein.

In some implementations, the representation of the amino acid sequence of the protein comprises one or more masked embeddings, and the method further comprises: processing a predicted amino acid sequence of the protein, defined by replacing each masked embedding in the representation of the amino acid sequence by a corresponding predicted embedding, using a protein folding neural network to generate data defining a predicted protein structure of the predicted amino acid sequence; and processing both: (i) the masked representation of the protein, and (ii) the predicted protein structure of the predicted amino acid sequence, using the protein reconstruction neural network to generate a new predicted embedding corresponding to one or more masked embeddings that are included in the masked representation of the protein.

In some implementations, each masked embedding included in the masked representation of the protein is a default embedding.

In some implementations, the default embedding comprises a vector of zeros.

In some implementations, each predicted embedding corresponding to a masked embedding in the representation of the structure of the protein defines a prediction for a spatial distance between a corresponding pair of amino acids in the structure of the protein.

In some implementations, at least one of the embeddings of the representation of the amino acid sequence of the protein is masked.

In some implementations, at least one of the embeddings of the representation of the structure of the protein is masked.

In some implementations, the representation of the amino acid sequence of the protein comprises a plurality of single embeddings that each correspond to a respective position in the amino acid sequence of the protein; the representation of the structure of the protein comprises a plurality of pair embeddings that each corresponding to a respective pair of positions in the amino acid sequence of the protein; the protein reconstruction neural network comprises a sequence of update blocks; each update block has a respective set of update block parameters and performs operations comprising: receiving current pair embeddings and current single embeddings; updating the current single embeddings, in accordance with values of the update block parameters of the update block, based on the current pair embeddings; and updating the current pair embeddings, in accordance with the values of the update block parameters of the update block, based on the updated single embeddings; and a final update block in the sequence of update blocks generates final pair embeddings and final single embeddings.

In some implementations, the protein reconstruction neural network performs further operations comprising, for each of one or more masked single embeddings in the representation of the amino acid sequence of the protein: generating the predicted embedding for the masked single embedding based on the corresponding final single embedding generated by the final update block.

In some implementations, the protein reconstruction neural network performs further operations comprising, for each of one or more masked pair embeddings in the representation of the amino acid sequence of the protein: generating the predicted embedding for the masked pair embedding based on the corresponding final pair embedding generated by the final update block.

In some implementations, updating the current single embeddings based on the current pair embeddings comprises: updating the current single embeddings using attention over the current single embeddings, wherein the attention is conditioned on the current pair embeddings.

In some implementations, updating the current single embeddings using attention over the current single embeddings comprises: generating, based on the current single embeddings, a plurality of attention weights; generating, based on the current pair embeddings, a respective attention bias corresponding to each of the attention weights; generating a plurality of biased attention weights based on the attention weights and the attention biases; and updating the current single embeddings using attention over the current single embeddings based on the biased attention weights.

In some implementations, updating the current pair embeddings based on the updated single embeddings comprises: applying a transformation operation to the updated single embeddings; and updating the current pair embeddings by adding a result of the transformation operation to the current pair embeddings.

In some implementations, the transformation operation comprises an outer product operation.

In some implementations, updating the current pair embeddings based on the updated single embeddings further comprises, after adding the result of the transformation operation to the current pair embeddings: updating the current pair embeddings using attention over the current pair embeddings, wherein the attention is conditioned on the current pair embeddings.

According to another aspect there is provided a method of obtaining a ligand, wherein the ligand is a drug or a ligand of an industrial enzyme, the method comprising: determining a predicted structure of a target protein by generating predicted embeddings that define a complete protein structure representation for the target protein, wherein the masked representation of the protein comprises a complete representation of the amino acid sequence of the target protein and wherein the representation of the structure of the protein comprises a fully masked representation of the structure of the target protein; evaluating an interaction of one or more candidate ligands with the predicted structure of the target protein; and selecting one or more of the candidate ligands as the ligand dependent on a result of the evaluating.

According to another aspect there is provided a method of obtaining a ligand, wherein the ligand is a drug or a ligand of an industrial enzyme, the method comprising: determining a predicted structure of each of a plurality of target proteins by generating predicted embeddings that define a complete protein structure representation for each target protein, wherein for each target protein the masked representation of the protein comprises a complete representation of the amino acid sequence of the target protein and wherein the representation of the structure of the protein comprises a fully masked representation of the structure of the target protein; evaluating the interaction of the one or more candidate ligands with the predicted structure of each of the target proteins; and selecting one or more of the candidate ligands as the ligand to either i) obtain a ligand that interacts with each of the target proteins, or ii) obtain a ligand that interacts with only one of the target proteins.

In some implementations, the target protein comprises a receptor or enzyme, and the ligand is an agonist or antagonist of the receptor or enzyme.

According to another aspect, there is provided a method of obtaining a polypeptide ligand, wherein the ligand is a drug or a ligand of an industrial enzyme, the method comprising: for each of one or more candidate polypeptide ligands, determining a predicted structure of the candidate polypeptide ligand by generating predicted embeddings that define a complete protein structure representation for the candidate polypeptide ligand, wherein for each of the one or more candidate polypeptide ligands the masked representation of the protein comprises a complete representation of the amino acid sequence of the candidate polypeptide ligand and wherein the representation of the structure of the protein comprises a fully masked representation of the structure of the candidate polypeptide ligand; obtaining a target protein structure of a target protein; evaluating an interaction between the predicted structure of each of the one or more candidate polypeptide ligands and the target protein structure; and selecting one of the one or more of the candidate polypeptide ligands as the polypeptide ligand dependent on a result of the evaluating.

In some implementations the target protein comprises a receptor or enzyme, and the ligand is an agonist or antagonist of the receptor or enzyme, or the polypeptide ligand comprises an antibody and the target protein comprises an antigen, and the antibody binds to the antigen to provide a therapeutic effect.

According to another aspect there is provided a method of obtaining an antibody for an antigen, the method comprising: determining a predicted structure and amino acid sequence of the antibody by generating predicted embeddings that define i) a complete amino acid sequence representation for the antibody, and ii) a complete protein structure representation for the antibody, wherein the masked representation of the protein includes a representation of a paratope of the antibody that binds to the antigen and comprises i) a partially masked representation of the amino acid sequence of the antibody, and ii) a partially masked representation of the structure of the antibody.

In some implementations, the antigen comprises a virus protein or a cancer cell protein.

According to another aspect there is provided a method of obtaining a diagnostic antibody marker of a disease, the method comprising: for each of one or more candidate antibodies, determining a predicted structure of the candidate antibody by generating predicted embeddings that define a complete protein structure representation for the candidate antibody, wherein for each of the one or more candidate antibodies the masked representation of the protein comprises a complete representation of the amino acid sequence of the candidate antibody and wherein the representation of the structure of the protein comprises a fully masked representation of the structure of the candidate antibody; obtaining a target protein structure of a target protein; evaluating an interaction between the predicted structure of each of the one or more candidate antibodies and the target protein structure; and selecting one of the one or more of the candidate antibodies as the diagnostic antibody marker dependent on a result of the evaluating.

According to another aspect there is provided a method of designing a mutated protein with an optimized property, comprising: obtaining i) a complete representation of the amino acid sequence of a known protein, and ii) a complete protein structure representation for the known protein; and for each of one or more candidate mutated proteins, determining a predicted amino acid sequence for the candidate mutated protein by generating predicted embeddings that define a complete amino acid sequence for the candidate mutated protein, wherein generating the predicted embeddings comprises: generating a partially masked representation of the candidate mutated protein by masking one or more of the embeddings in the representation of the amino acid sequence of the candidate mutated protein; generating, for each masked amino acid embedding, a respective score distribution that defines a score for each amino acid type in a set of possible amino acid types; generating the predicted embedding by sampling a respective type for each masked amino acid in accordance with the score distribution for the amino acid; and selecting one of the candidate mutated proteins as the mutated protein by identifying from amongst the candidate mutated proteins the predicted amino acid sequence that predicts the optimum property for the candidate mutated protein.

In some implementations, the method further comprises synthesizing the mutated protein.

According to another aspect there is provided a method of identifying the presence of a protein mis-folding disease, comprising: determining a predicted structure of a protein by generating predicted embeddings that define a complete protein structure representation for the protein, wherein the masked representation of the protein comprises a complete representation of the amino acid sequence of the protein and wherein the representation of the structure of the protein comprises a fully masked representation of the structure of the protein; obtaining a structure of a version of the protein obtained from a human or animal body; comparing the predicted structure of the protein with the structure of a version of the protein obtained from a human or animal body; and identifying the presence of a protein mis-folding disease dependent upon a result of the comparison.

According to another aspect there is provided a method of obtaining the amino acid sequence of a protein, comprising: receiving a structure of the protein, wherein the structure of the protein has been obtained by experiment; determining a complete protein structure representation for the protein from the structure; and determining a predicted amino acid sequence of the protein by generating predicted embeddings that define a complete amino acid sequence representation for the protein, wherein the masked representation of the protein comprises a complete representation of the structure of the protein, wherein the representation of the amino acid sequence of the protein comprises a fully masked representation of the amino acid sequence of the protein, and wherein the predicted amino acid sequence of the protein is the obtained amino acid sequence of the protein.

According to another aspect there is provided a system comprising: one or more computers; and one or more storage devices communicatively coupled to the one or more computers, wherein the one or more storage devices store instructions that, when executed by the one or more computers, cause the one or more computers to perform operations of the methods described herein.

According to another aspect there are provided one or more non-transitory computer storage media storing instructions that when executed by one or more computers cause the one or more computers to perform operations of the methods described herein.

Particular embodiments of the subject matter described in this specification can be implemented so as to realize one or more of the following advantages.

Generally, protein folding (i.e., predicting protein structures from amino acid sequences) and protein design (i.e., predicting amino acid sequences from protein structures) are closely related tasks. The system described in this specification can be trained to perform both of these tasks in parallel. In particular, the system can be provided with a masked representation of a protein that includes a representation of an amino acid sequence of the protein and a representation of the structure of the protein, where one or both of these representations is at least partially masked. The system then processes the masked protein representation to generate a “complete” (i.e., unmasked) representation of the protein, i.e., that includes predictions for masked portions of the amino acid sequence representation and the protein structure representation. As a result of being trained to perform both protein folding and protein design in parallel, the system can achieve a higher prediction accuracy on each of these tasks than if the system had been trained to perform either of these tasks independently of the other. The system can, in some cases, achieve an acceptable prediction accuracy on protein folding tasks, protein design tasks, or both, while consuming fewer computational resources (e.g., memory and computing power) than other systems that perform either of these tasks independently of the other.

The system described in this specification can unmask a masked protein representation by incrementally replacing masked embeddings in the masked protein representation with corresponding predicted embeddings over a sequence of iterations. Replacing the masked embeddings in the masked protein representation with corresponding predicted embeddings over a sequence of iterations rather than, e.g., all at once in a single iteration, can enable the system to incrementally accumulate contextual information and thereby unmask the masked protein representation with higher accuracy.

The system described in this specification can, at each of one or more iterations, predict the protein structure of a current amino acid sequence that is defined by replacing each masked embedding in the amino acid sequence representation by a corresponding predicted embedding generated at the current iteration. The system can then process both the predicted protein structure and the masked protein representation at the next iteration, which can enable the system to adaptively correct errors in the predicted embeddings that cause the corresponding predicted protein structure to deviate from the target protein structure representation. In particular, at each iteration after the first iteration, the system can generate new (and potentially corrected) predicted embeddings at the iterations based at least in part on the predicted protein structure generated at the previous iteration.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example protein reconstruction system.

FIG. 2 shows an example architecture of a protein reconstruction neural network.

FIG. 3 shows an example architecture of an update block of the protein reconstruction neural network.

FIG. 4 shows an example architecture of a single embedding update block.

FIG. 5 shows an example architecture of a pair embedding update block.

FIG. 6 is a flow diagram of an example process for unmasking a masked representation of a protein using a protein reconstruction neural network.

Like reference numbers and designations in the various drawings indicate like elements.

### DETAILED DESCRIPTION

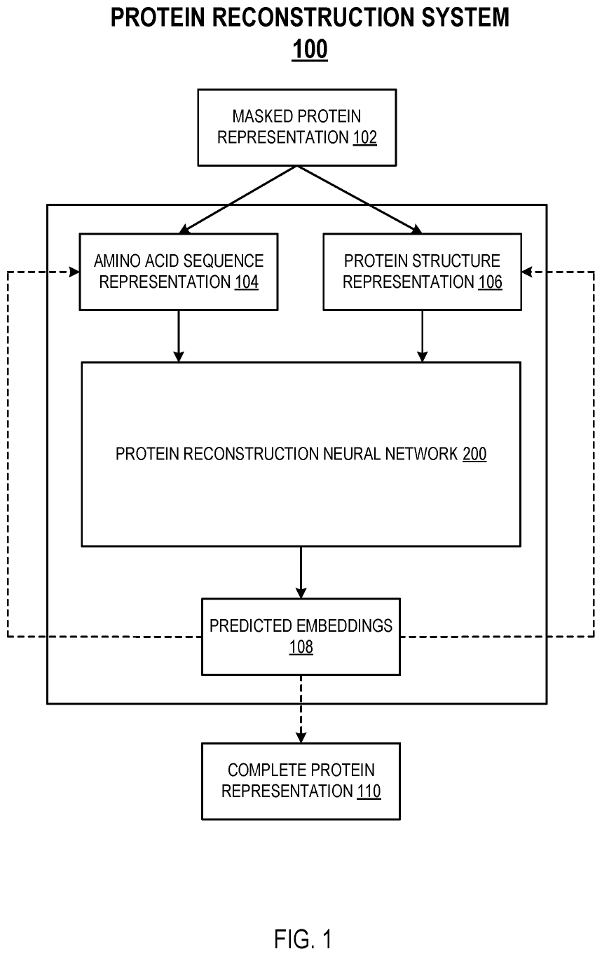

FIG. 1 shows an example protein reconstruction system 100. The protein reconstruction system 100 is an example of a system implemented as computer programs on one or more computers in one or more locations in which the systems, components, and techniques described below are implemented.

The system 100 is configured to receive a masked representation of a protein 102 that includes: (i) a representation of the amino acid sequence of the protein (i.e., the amino acid sequence representation 104), and (ii) a representation of the structure of the protein (i.e., the protein structure representation 106). The amino acid sequence representation 104 and the protein structure representation 106 are each represented by respective collections of embeddings, and at least one of the embeddings of the amino acid sequence representation 104, the protein structure representation 106, or both, is masked. An embedding can be referred to as being “masked,” e.g., if the embedding is a default (e.g., predefined) embedding, e.g., an embedding represented as a vector of zeros.

The amino acid sequence representation 104 can include a respective embedding corresponding to each position in the amino acid sequence of the protein. Each embedding of the amino acid sequence representation 104 that is not a masked embedding can represent the amino acid at the corresponding position in the amino acid sequence, e.g., by a one-hot embedding that identifies the amino acid from a set of possible amino acids. The set of possible amino acids can include, e.g., alanine, arginine, asparagine, etc., and the total number of amino acid in the set of possible amino acids can be, e.g., 20.

The protein structure representation 106 can include a respective embedding corresponding to each “structural feature” in a set of structural features that characterize the protein structure.

For example, each structural feature in the set of structural features characterizing the protein structure can define the spatial distance (e.g., measured in Angstroms) separating specified atoms (e.g., alpha carbon atoms) in a corresponding pair of amino acids in the protein structure. In this example, an embedding representing the spatial distance between a pair of amino acids in the protein structure can be a one-hot embedding that identifies the spatial distance between the pair of amino acids as being included in one distance interval from a set of possible distance intervals. The set of possible distance intervals can be, e.g., 0-2 Angstroms, 2-4 Angstroms, 4-6 Angstroms, etc.

As another example, each structural feature in the set of structural features characterizing the protein structure can define the spatial location of an atom (e.g., an alpha carbon atom) in a corresponding amino acid in the protein structure. Each embedding of the protein structure representation that is not a masked embedding can represent the spatial location of an atom in a corresponding amino acid in the protein structure, e.g., as an x-y-z coordinate in a predefined Cartesian coordinate system. As a further example, the structural features can define backbone atom torsion angles of the amino acid residues in the protein.

Certain embeddings in the amino acid sequence representation 104 and the protein structure representation 106 can be masked, e.g., because they represent information about the protein that is not known. For example, if the amino acid sequence of the protein is known but the structure of the protein is unknown, then the amino acid sequence representation can be “complete” (i.e., with none of the embeddings being masked), while all of the embeddings of the protein structure representation can be masked. As another example, if the structure of the protein is known but the amino acid sequence of the protein is unknown, then the protein structure representation can be complete, while all of the embeddings of the amino acid sequence representation can be masked. As another example, if both the amino acid sequence of the protein and the structure of the protein are only partially known, then both the amino acid sequence representation and the protein structure representation can include some embeddings that are masked and others that are not masked.

The system 100 processes the amino acid sequence representation 104 and the protein structure representation 106 using a protein reconstruction neural network 200 to generate a respective predicted embedding corresponding to each masked embedding in the masked protein representation 102. A predicted embedding 108 corresponding to a masked embedding in the amino acid sequence representation 104 can define a prediction of the identity of the amino acid at a corresponding position in the amino acid sequence of the protein. A predicted embedding 108 corresponding to a masked embedding in the protein structure representation 106 can define a prediction for a corresponding structural feature of the protein, e.g., the spatial distance between respective atoms in a corresponding pair of amino acids in the protein. Generating the predicted embeddings 108 can be understood as reconstructing the masked embeddings in the masked protein representation 102 using the contextual information available from the non-masked embeddings in the masked protein representation 102.

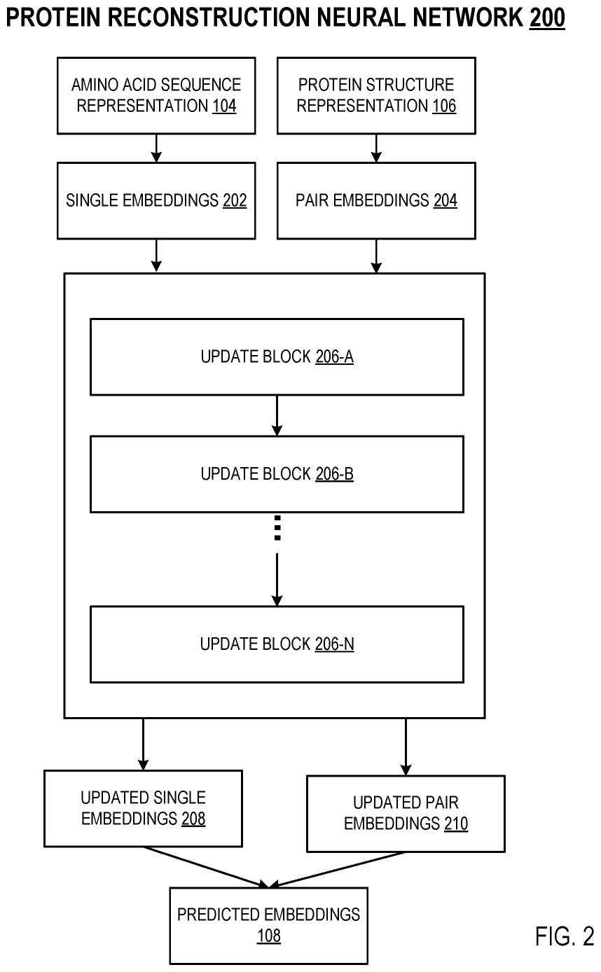

The protein reconstruction neural network 200 can have any appropriate neural network architecture that enables it to perform its described functions, including any appropriate neural network layers (e.g., fully-connected layers, convolutional layers, attention layers, etc.) arranged in any appropriate configuration (e.g., as a sequential sequence of layers). An example architecture of the protein reconstruction neural network 200 is described in more detail with reference to FIG. 2-FIG. 5. However existing protein reconstruction neural networks may also be adapted to use the described techniques, i.e. to jointly process representations of both the amino acid sequence and the protein structure e.g. iteratively.

Replacing the masked embeddings in the masked protein representation 102 with the corresponding predicted embeddings 108 yields a complete protein representation 110, i.e., such that none of the embeddings in the complete protein representation 110 are masked. That is, the complete protein representation can define a complete reconstruction of the amino acid sequence of the protein (i.e., where the identity of the amino acid at each position in the amino acid sequence is specified and not masked), and a complete reconstruction of the protein structure (i.e., where each structural feature in the set of structural features characterizing the protein structure is specified and not masked). The system 100 can then provide the complete protein representation 110, or a portion thereof (e.g., only the complete amino acid sequence representation, or only the complete protein structure representation), as an output.

In some implementations, the system 100 incrementally replaces the masked embeddings in the masked protein representation 102 with corresponding predicted embeddings 108 over a sequence of iterations. More specifically, at each iteration, the system 100 processes the current masked protein representation 102 using the protein reconstruction neural network 200 to generate predicted embeddings 108, and updates the current masked protein representation 102 by replacing one or more of the remaining masked embeddings by corresponding predicted embeddings 108. The number of remaining masked embeddings in the masked protein representation 102 is reduced at each iteration, and at the last iteration, the system 100 replaces all remaining masked embeddings in the masked protein representation 102 with corresponding predicted embeddings 108 generated at the last iteration.

The system 100 can determine which masked embeddings in the masked protein representation 102 are to be replaced by corresponding predicted embeddings 108 at each iteration in any of a variety of ways; a few examples follow.

In one example, at each iteration, the system 100 can randomly select a predefined fraction (e.g., 15%) of the remaining masked embeddings in the masked protein representation 102 to be replaced by corresponding predicted embeddings 108. When the system 100 determines that fewer than a predefined threshold number of masked embeddings remain in the masked protein representation 102, the system 100 can replace all the remaining masked embeddings with corresponding predicted embeddings 108 and terminate the iterative process.

In another example, at each iteration, the system 100 can determine which masked embeddings in the amino acid sequence representation 104 to replace by corresponding predicted embeddings 108 based on an arrangement of the embeddings of the amino acid sequence representation 104 into an array. More specifically, the embeddings of the amino acid sequence representation 104 can be associated with an arrangement into a one-dimensional (1D) array, where the embedding at position i in the array corresponds to the amino acid at position i in the amino acid sequence of the protein. At each iteration, the system 100 can determine that a masked embedding of the amino acid sequence representation 104 should be replaced by a corresponding predicted embedding 108 if the masked embedding is adjacent to a non-masked embedding in the 1D array of embeddings of the amino acid sequence representation.

In another example, at each iteration, the system 100 can determine which masked embeddings in the protein structure representation 106 to replace by corresponding predicted embeddings 108 based on an arrangement of the embeddings of the protein structure representation into an array. More specifically, the embeddings of the protein structure representation 106 can be associated with an arrangement into a two-dimensional (2D) array, where the embedding at position (i,j) in the array corresponds to the pair of amino acids at positions i and j in the amino acid sequence of the protein. At each iteration, the system 100 can determine that a masked embedding of the protein structure representation 106 should be replaced by a corresponding predicted embedding 108 if the masked embedding is adjacent to a non-masked embedding in the 2D array of embeddings of the protein structure representation 106. One embedding can be understood as being “adjacent” to another embedding in a 2-D array of embeddings, e.g., if they are adjacent in the same row of the 2-D array, or adjacent in the same column of the 2-D array.

Replacing the masked embeddings in the masked protein representation 102 with corresponding predicted embeddings 108 over a sequence of iterations (rather than, e.g., all at once) can enable the system 100 to incrementally accumulate contextual information and thereby generate more accurate predicted embeddings 108.

In some implementations, the amino acid sequence representation 104 includes at least one masked embedding, and at each of one or more iterations, the system 100 generates a respective predicted embedding 108 corresponding to each masked embedding in the amino acid sequence representation 104. For convenience, an amino acid sequence defined by replacing each masked embedding in the amino acid sequence representation 104 with the corresponding predicted embedding 108 generated at a current iteration will be referred to as the “current amino acid sequence.” At each iteration, the system 100 can process the current amino acid sequence using a protein folding neural network to generate a predicted structure of a protein having the current amino acid sequence. The system can then provide the predicted protein structure as an additional input to the protein reconstruction neural network 200 at the next iteration.

To provide the predicted protein structure as an additional input to the protein reconstruction neural network 200 at the next iteration, the system 100 can generate a representation of the predicted protein structure. The representation of the predicted protein structure can include a respective embedding corresponding to each structural feature in a set of structural features that characterize the predicted protein structure. For example, the representation of the predicted protein structure can include respective embeddings representing spatial distances between pairs of amino acids in the predicted protein structure, as described above. The protein reconstruction neural network 200 can process the additional input defined by the representation of the predicted protein structure in any appropriate way. For example, the protein reconstruction neural network 200 can sum, average, or otherwise combine the representation of the predicted protein structure with the protein structure representation 106. The protein reconstruction neural network 200 can then process the resulting combined protein structure representation and the amino acid sequence representation 104 in accordance with the parameter values of the protein reconstruction neural network 200 to generate predicted embeddings 108 for the next iteration, as described above.

The protein folding neural network can have any appropriate neural network architecture that enables it to perform its described function, i.e., processing an input including a representation of an amino acid sequence to generate a predicted structure of a protein having the amino acid sequence. In particular, the protein folding neural network can include any appropriate neural network layers (e.g., fully-connected layers, convolutional layers, attention layers, etc.) arranged in any appropriate configuration (e.g., as a sequence of layers).

Providing the predicted protein structure corresponding to the current amino acid sequence to the protein reconstruction neural network 200 can enable the system 100 implicitly compare the predicted protein structure and the protein structure representation 106. This comparison can enable the protein reconstruction neural network 200 to correct potential errors in the current amino acid sequence that cause the corresponding predicted protein structure to deviate from the protein structure representation 106, thereby improving the performance (e.g., prediction accuracy) of the system 100.

The system 100 can generate a predicted protein structure corresponding to the current amino acid sequence at each iteration and provide it to the reconstruction neural network at the next iteration as an alternative to, or in combination with, incrementally replacing the masked embeddings in the masked protein representation at each iteration. That is, at each iteration, the system can do one or both of: (i) process a (temporary) amino acid sequence defined by replacing each masked embedding in the amino acid sequence representation 104 with a corresponding predicted embedding 108 generated at the iteration to generate a corresponding predicted protein structure that is provided to the reconstruction neural network at the next iteration, and (ii) use one or more predicted embeddings generated at the iteration to replace corresponding masked embeddings in the masked protein representation (e.g., masked embeddings in the amino acid sequence representation 104, the protein structure representation 106, or both).

A few examples of possible applications of the system 100 are described in more detail next.

In one example, the system 100 can be used to predict the protein structure corresponding to a known amino acid sequence by processing a complete amino acid sequence representation and a fully masked protein structure representation to “unmask” the protein structure representation. Unmasking the protein structure representation refers to generating predicted embeddings that define the complete protein structure representation.

In another example, the system 100 can be used to predict the amino acid sequence corresponding to a known protein structure by processing a complete protein structure representation and a fully masked amino acid sequence representation to “unmask” the amino acid sequence representation. Unmasking the amino acid sequence representation refers to generating predicted embeddings that define the complete amino acid sequence representation. The known protein structure may be obtained by experiment using conventional physical techniques e.g. x-ray crystallography, magnetic resonance techniques, or cryogenic electron microscopy (cryo-EM).

In another example, the system 100 can be used to generate a complete protein representation for a protein with a partially known amino acid sequence and a partially known protein structure. In particular, the system can process a partially masked amino acid sequence representation representing the partially known amino acid sequence and a partially masked protein structure representation representing the partially known protein structure to unmask the amino acid sequence representation and the protein structure representation. Generating complete protein representations from partially masked amino acid sequences and partially masked protein structures can be performed, e.g., to design a full antibody starting from a known paratope, e.g., that selectively binds to a particular antigen, in particular to provide a therapeutic effect. For example the antigen may comprise a virus protein or a cancer cell protein. The designed antibody may then be synthesized.

To design a full antibody starting from a known paratope, the system 100 can be used to process a partially masked representation of the amino acid sequence of the antibody and a partially masked representation of the structure of the antibody to generate a complete representation of the antibody. The representation of the amino acid sequence of the antibody can include one-hot embeddings representing the known amino acids of the paratope, and masked amino acid embeddings for each other amino acid in the antibody. The representation of the protein structure of the antibody can include embeddings representing the structure of the paratope, and masked embeddings representing the structure of the remainder of the antibody (i.e., outside the paratope). The complete representation of the antibody can define the respective type of each amino acid in the antibody, as well as the structure of the antibody.

In another example, the system 100 can be used to generate a complete protein representation for a protein with: (i) a partially known amino acid sequence and a fully known protein structure, or (ii) a fully known amino acid sequence and a partially known protein structure. For example, the system can process a partially masked amino acid sequence representation and a complete protein structure representation to unmask the amino acid sequence representation.

Generating a complete protein representation from a partially masked amino acid sequence of a protein and a complete structure of the protein can be performed, e.g., to optimize certain characteristics of the protein, e.g., binding affinity, solubility, stability, aggregation propensity, or any other appropriate characteristics. For example, starting from a protein with a known amino acid sequence and a known protein structure, a masked representation of the amino acid sequence of the protein can be generated, i.e., where the identities of one or more amino acids in the protein are masked. The system 100 can process the masked amino acid sequence representation and a complete structure representation for the protein to generate, for each masked amino acid, a respective score distribution that defines a score for each amino acid type in a set of possible amino acid types. An example of generating a score distribution over amino acid types is described later. The system 100 can then generate multiple “candidate” proteins, where the amino acid sequence of each candidate protein is determined by sampling a respective type for each masked amino acid in accordance with the score distribution for the amino acid. The value of a respective property (e.g., solubility, stability, binding affinity, or aggregation propensity) can be predicted for each candidate protein, and the candidate protein having the most desirable (e.g., highest or lowest) value of the respective property can be selected. The value of the respective property may be predicted from amino acid sequence of the candidate protein using e.g. published techniques or available software tools. The selected candidate protein can be understood as “mutating” the original protein to optimize a desired property of the protein (e.g., solubility, stability, or binding affinity). Thus a mutated protein with the desired property may be synthesized by synthesizing a protein with the amino acid sequence of the selected candidate protein.

The system 100 can receive the masked protein representation 102, e.g., from a remotely located user of the protein reconstruction system 100 through an interface (e.g., an application programming interface (API)) made available by the protein reconstruction system 100 by way of a data communications network (e.g., the internet). After generating the complete protein representation 110, the system 100 can provide the complete protein representation 110 (or a portion thereof) to the remotely located user by way of the data communications network.

A training engine can train the parameters of the protein reconstruction neural network 200 on a set of training examples over multiple training iterations. Each training example can define a complete protein representation, i.e., that includes a complete amino acid sequence representation and a complete protein structure representation of a protein.

At each training iteration, the training engine can sample one or more complete protein representations and generate a masked protein representation corresponding to each complete protein representation, e.g., by randomly masking portions of the complete protein representation. The training engine can process each masked protein representation using the system 100, in accordance with the current parameter values of the protein reconstruction neural network (as described above), to generate a respective predicted embedding for each masked embedding of the masked protein representation. The training engine can then determine gradients, with respect to the parameters of the protein reconstruction neural network, of an objective function that measures an error between: (i) the predicted embeddings generated by the system 100, and (ii) the corresponding embeddings defined by the complete protein representations. The training engine can measure the error between a predicted embedding generated by the system 100 and a corresponding embedding from a complete protein representation, e.g., by a cross-entropy loss or a squared-error loss. The training engine use the gradients of the objective function to update the parameter values of the protein reconstruction neural network using any appropriate the update rule of any appropriate gradient descent optimization technique, e.g., RMSprop or Adam.

FIG. 2 shows an example architecture of a protein reconstruction neural network 200. The protein reconstruction neural network 200 is configured to process a masked representation of a protein that includes: (i) an amino acid sequence representation 104, and (ii) a protein structure representation 106, where one or more of the embeddings of the masked protein representation are masked.

The amino acid sequence representation 104 includes a respective “single” embedding corresponding to each position in the amino acid sequence of the protein. Each embedding of the amino acid sequence representation 104 that is not a masked embedding can represent the amino acid at the corresponding position in the amino acid sequence, e.g., by a one-hot embedding that identifies the amino acid from a set of possible amino acids. The protein reconstruction neural network 200 can optionally apply position encoding data to each single embedding, where the positional encoding data applied to a single embedding is a function of the index of the position in the amino acid sequence corresponding to the single embedding. For example, the protein reconstruction neural network 200 can apply sinusoidal positional encoding data to each single embedding, as described with reference to A. Vaswani et al., “Attention is all you need,” 21st Conference on Neural Informational Processing Systems (NIPS 2017).

The protein structure representation 106 includes a respective “pair” embedding corresponding to each pair of amino acids in the protein (e.g. N×N pairs). Each pair embedding that is not a masked embedding can represent the spatial distance between a corresponding pair of amino acids, e.g., by a one-hot embedding that identifies the spatial distance between the pair of amino as being included in one distance interval from a set of possible distance intervals.

The protein reconstruction neural network 200 includes a sequence of update blocks 206-A-N. Throughout this specification, a “block” refers to a portion of a neural network, e.g., a subnetwork of the neural network that includes one or more neural network layers.

Each update block in the protein reconstruction neural network is configured to receive a block input that includes a set of single embeddings and a set of pair embeddings, and to process the block input to generate a block output that includes updated single embeddings and updated pair embeddings.

The protein reconstruction neural network 200 provides the single embeddings 202 and the pair embeddings 204 included in the network input of the protein reconstruction neural network 200 to the first update block (i.e., in the sequence of update blocks). The first update block processes the single embeddings 202 and the pair embeddings 204 to generate updated single embeddings and updated pair embeddings.

For each update block after the first update block, the protein reconstruction neural network 200 provides the update block with the single embeddings and the pair embeddings generated by the preceding update block, and provides the updated single embeddings and the updated pair embeddings generated by the update block to the next update block.

The protein reconstruction neural network 200 gradually enriches the information content of the single embeddings 202 and the pair embeddings 204 by repeatedly updating them using the sequence of update blocks 206-A-N.

The final update block in the sequence of update blocks outputs a set of updated single embeddings 208 and a set of updated pair embeddings 210. Each updated single embedding 208 can include a respective “soft” score for each amino acid in the set of possible amino acids, and each updated pair embedding can include a respective “soft” score for each distance interval in the set of possible distance intervals.

The protein reconstruction neural network 200 can identify the predicted embedding 108 for a masked single embedding from the amino acid sequence representation 104 as being a one-hot embedding representing the amino acid that is associated with the highest soft score by the corresponding updated single embedding 208. Similarly, the protein reconstruction neural network 200 can identify the predicted embedding 108 for a masked pair embedding from the protein structure representation 106 as being a one-hot embedding representing the distance interval that is associated with the highest soft score by the corresponding updated pair embedding 210.

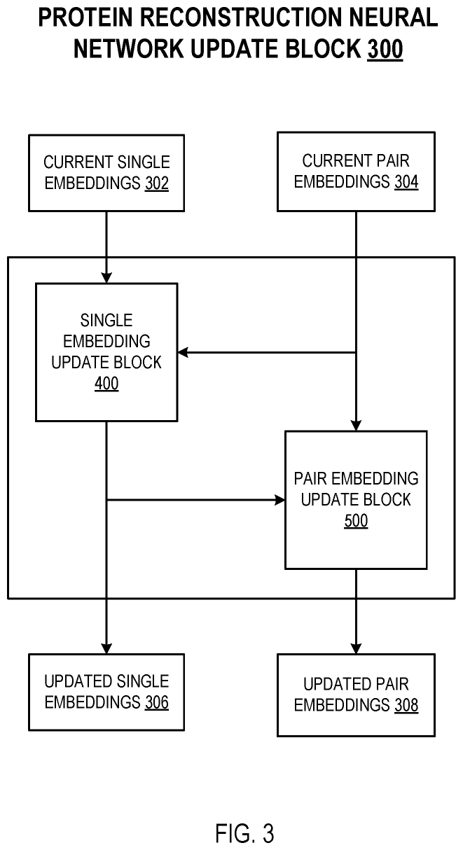

FIG. 3 shows an example architecture of an update block 300 of the protein reconstruction neural network 200, i.e., as described with reference to FIG. 2.

The update block 300 receives a block input that includes the current single embeddings 302 and the current pair embeddings 304, and processes the block input to generate the updated single embeddings 306 and the updated pair embeddings 308.

The update block 300 includes a single embedding update block 400 and a pair embedding update block 500.

The single embedding update block 400 updates the current single embeddings using the current pair embeddings 304, and the pair embedding update block 500 updates the current pair embeddings 304 using the updated single embeddings (i.e., that are generated by the single embedding update block 400).

Generally, the single embeddings and the pair embeddings can encode complementary information. The single embedding update block 400 enriches the information content of the single embeddings using complementary information encoded in the pair embeddings, and the pair embedding update block 500 enriches the information content of the pair embeddings using complementary information encoded in the single embeddings. As a result of this enrichment, the updated single embeddings and the updated pair embedding encode information that is more relevant to accurately unmasking the masked embeddings of the masked protein representation.

The update block 300 is described herein as first updating the current single embeddings 302 using the current pair embeddings 304, and then updating the current pair embeddings 304 using the updated single embeddings 306. The description should not be understood as limiting the update block to performing operations in this sequence, e.g., the update block could first update the current pair embeddings using the current single embeddings, and then update the current single embeddings using the updated pair embeddings.

The update block 300 is described herein as including a single embedding update block 400 (i.e., that updates the current single embeddings) and a pair embedding update block 500 (i.e., that updates the current pair embeddings). The description should not be understood to limiting the update block 300 to include only one single embedding update block or only one pair embedding update block. For example, the update block 300 can include multiple single embedding update blocks that update the single embeddings multiple times before the single embeddings are provided to a pair update block for use in updating the current pair embeddings. As another example, the update block 300 can include multiple pair update blocks that update the pair embeddings multiple times using the single embeddings.

The single embedding update block 400 and the pair embedding update block 500 can have any appropriate architectures that enable them to perform their described functions.

In some implementations, the single embedding update block 400, the pair embedding update block 500, or both, include one or more “self-attention” blocks. As used throughout this document, a self-attention block generally refers to a neural network block that updates a collection of embeddings, i.e., that receives a collection of embeddings and outputs updated embeddings. To update a given embedding, the self-attention block can determine a respective “attention weight”, e.g. a similarity measure, between the given embedding and each of one or more selected embeddings e.g. the received collection of embeddings, and then update the given embedding using: (i) the attention weights, and (ii) the selected embeddings. For example an updated embedding may comprise a sum of values each derived from one of the selected embeddings and each weighted by a respective attention weight. For convenience, the self-attention block may be said to update the given embedding using attention “over” the selected embeddings.

For example, a self-attention block may receive a collection of input embeddings {xᵢ}ᵢ₌₁^{N}, where N is the number of amino acids in the protein, and to update embedding xᵢ, the self-attention block may determine attention weights [a_{i,j}]ⱼ₌₁^{N} where a_{i,j} denotes the attention weight between xᵢ and xⱼ, as:

$${[ {a}_{i , j} ]}_{j = 1}^{N} = softmax ( \frac{( {W}_{q} {x}_{i} ) {K}^{T}}{c} ) \qquad ( 1 )$$

$${K}^{T} = {[ {W}_{k} {x}_{j} ]}_{j = 1}^{N} \qquad ( 2 )$$

where W_{q} and Wₖ are learned parameter matrices, softmax (·) denotes a soft-max normalization operation, and c is a constant. Using the attention weights, the self-attention layer may update embedding xᵢ as:

$${x}_{i} ← {\sum }_{j = 1 … N} {a}_{i , j} \cdot ( {W}_{v} {x}_{j} ) \qquad ( 3 )$$

where Wᵥ is a learned parameter matrix. (W_{q}xᵢ can be referred to as the “query embedding” for input embedding xᵢ, Wₖxⱼ can be referred to as the “key embedding” for input embedding xᵢ, and Wᵥxⱼ can be referred to as the “value embedding” for input embedding xᵢ).

The parameter matrices W_{q} (the “query embedding matrix”), Wₖ (the “key embedding matrix”), and Wᵥ (the “value embedding matrix”) are trainable parameters of the self-attention block. The parameters of any self-attention blocks included in the single embedding update block 400 and the pair embedding update block 500 can be understood as being parameters of the update block 300 that can be trained as part of the end-to-end training of the protein reconstruction system 100 described with reference to FIG. 1. Generally, the (trained) parameters of the query, key, and value embedding matrices are different for different self-attention blocks, e.g., such that a self-attention block included in the single embedding update block 400 can have different query, key, and value embedding matrices with different parameters than a self-attention block included in the pair embedding update block 500.

In some implementations, the single embedding update block 400, the pair embedding update block 500, or both, include one or more self-attention blocks that are conditioned on (dependent upon) the pair embeddings, i.e., that implement self-attention operations that are conditioned on the pair embeddings. To condition a self-attention operation on the pair embeddings, the self-attention block can process the pair embeddings to generate a respective “attention bias” corresponding to each attention weight; each attention weight may then be biased by the corresponding attention bias. For example, in addition to determining the attention weights [a_{i,j}]ⱼ₌₁^{N} in accordance with equations (1)-(2), the self-attention block can generate a corresponding set of attention biases [b_{i,j}]ⱼ₌₁^{N}, where b_{i,j} denotes the attention bias between xᵢ and xⱼ. The self-attention block can generate the attention bias b_{i,j} by applying a learned parameter matrix to the pair embedding h_{i,j}, i.e., for the pair of amino acids in the protein indexed by (i,j).

The self-attention block can determine a set of “biased attention weights” [c_{i,j}]=ⱼ₌₁^{N}, where c_{i,j} denotes the biased attention weight between xᵢ and xⱼ, e.g., by summing (or otherwise combining) the attention weights and the attention biases. For example, the self-attention block can determine the biased attention weight c_{i,j} between embeddings xᵢ and xⱼ as:

c_{i,j}=a_{i,j}+b_{i,j}

where a_{i,j} is the attention weight between xᵢ and xⱼ and b_{i,j} is the attention bias between xᵢ and xⱼ. The self-attention block can update each input embedding xᵢ using the biased attention weights, e.g.:

$${x}_{i} ← {\sum }_{j = 1 … N} {c}_{i , j} \cdot ( {W}_{v} {x}_{j} ) \qquad ( 4 )$$

where Wᵥ is a learned parameter matrix.

Generally, the pair embeddings encode information characterizing the structure of the protein and the relationships between the pairs of amino acids in the structure of the protein. Applying a self-attention operation that is conditioned on the pair embeddings to a set of input embeddings allows the input embeddings to be updated in a manner that is informed by the protein structural information encoded in the pair embeddings. The update blocks of the protein reconstruction neural network can use the self-attention blocks that are conditioned on the pair embeddings to update and enrich the single embeddings and the pair embeddings themselves.

Optionally, a self-attention block can have multiple “heads” that each generate a respective updated embedding corresponding to each input embedding, i.e., such that each input embedding is associated with multiple updated embeddings. For example, each head may generate updated embeddings in accordance with different values of the parameter matrices W_{q}, Wₖ, and Wᵥ that are described with reference to equations (1)-(4). A self-attention block with multiple heads can implement a “gating” operation to combine the updated embeddings generated by the heads for an input embedding, i.e., to generate a single updated embedding corresponding to each input embedding. For example, the self-attention block can process the input embeddings using one or more neural network layers (e.g., fully connected neural network layers) to generate a respective gating value for each head. The self-attention block can then combine the updated embeddings corresponding to an input embedding in accordance with the gating values. For example, the self-attention block can generate the updated embedding for an input embedding xᵢ as:

$${\sum }_{k = 1}^{K} {a}_{k} \cdot {x}_{i}^{next} \qquad ( 5 )$$

where k indexes the heads, αₖ is the gating value for head k, and xᵢ^{next} is the updated embedding generated by head k for input embedding xᵢ.

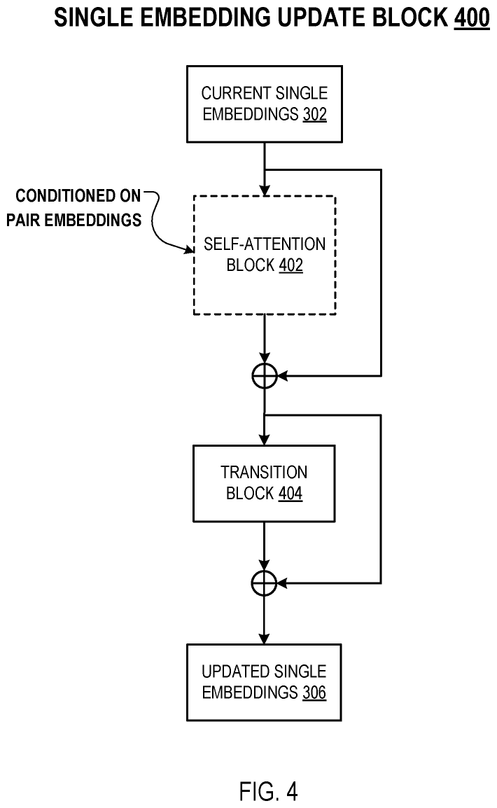

An example architecture of a single embedding update block 400 that uses self-attention blocks conditioned on the pair embeddings is described with reference to FIG. 4.

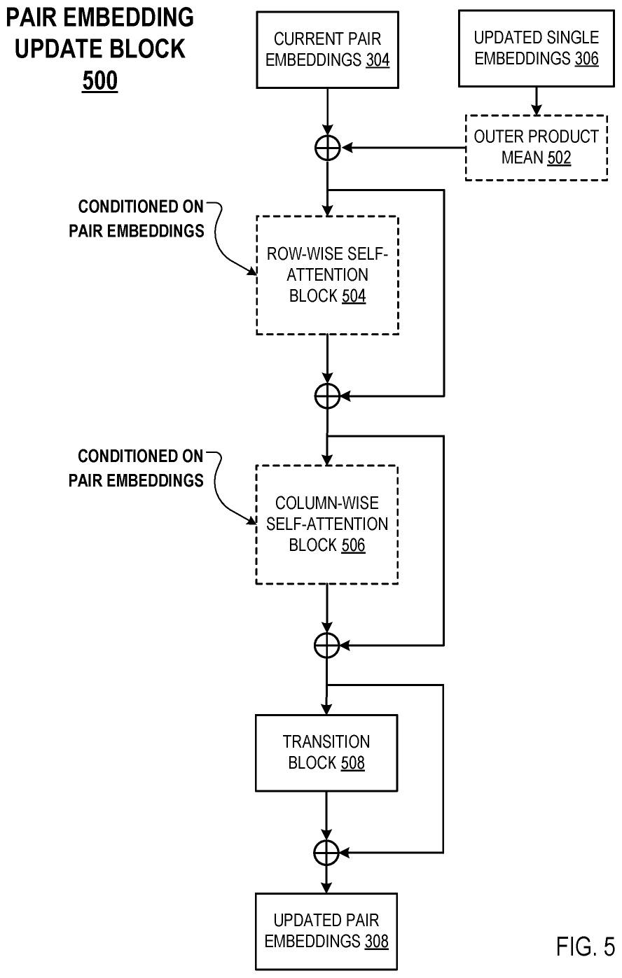

An example architecture of a pair embedding update block 500 that uses self-attention blocks conditioned on the pair embeddings is described with reference to FIG. 5. The example pair embedding update block described with reference to FIG. 5 updates the current pair embeddings based on the updated single embeddings by computing an outer product (hereinafter referred to as an outer product mean) of the updated single embeddings, adding the result of the outer product mean to the current pair embeddings (projected to the pair embedding dimension, if necessary), and processing the current pair embeddings using self-attention blocks that are conditioned on the current pair embeddings.

FIG. 4 shows an example architecture of a single embedding update block 400. The single embedding update block 400 is configured to receive the current single embeddings 302, and to update the current single embeddings 302 based (at least in part) on the current pair embeddings.

To update the current single embeddings 302, the single embedding update block 400 updates the single embeddings using a self-attention operation that is conditioned on the current pair embeddings. More specifically, the single embedding update block 400 provides the single embeddings to a self-attention block 402 that is conditioned on the current pair embeddings, e.g., as described with reference to FIG. 3, to generate updated single embeddings. Optionally, the single embedding update block can add the input to the self-attention block 402 to the output of the self-attention block 402. Conditioning the self-attention block 402 on the current pair embeddings enables the single embedding update block 400 to enrich the current single embeddings 302 using information from the current pair embeddings.

The single embedding update block then processes the current single embeddings 302 using a transition block, e.g., that applies one or more fully-connected neural network layers to the current single embeddings. Optionally, the single embedding update block 400 can add the input to the transition block 404 to the output of the transition block 404.

The single embedding update block can output the updated single embeddings 306 resulting from the operations performed by the self-attention block 402 and the transition block 404.

FIG. 5 shows an example architecture of a pair embedding update block 500. The pair embedding update block 500 is configured to receive the current pair embeddings 304, and to update the current pair embeddings 304 based (at least in part) on the updated single embeddings 306.

In the description which follows, the pair embeddings can be understood as being arranged into an N×N array, i.e., such that the embedding at position (i,j) in the array is the pair embedding corresponding to the amino acids at positions i and j in the amino acid sequence.

To update the current pair embeddings 304, the pair embedding update block 500 applies an outer product mean operation 502 to the updated single embeddings 306 and adds the result of the outer-product mean operation 502 to the current pair embeddings 304.

The outer product mean operation defines a sequence of operations that, when applied to the set of single embeddings represented as an 1×N array of embeddings, generates an N×N array of embeddings, i.e, where N is the number of amino acids in the protein. The current pair embeddings 304 can also be represented as an N×N array of pair embeddings, and adding the result of the outer product mean 502 to the current pair embeddings 304 refers to summing the two N×N arrays of embeddings.

To compute the outer product mean, the pair embedding update block generates a tensor A(·), e.g., given by:

A(res1,res2,ch1,ch2)=LeftAct(res1,ch1)·RightAct(res2,ch2) (6)

where res1, res2∈{1, . . . , N}, ch1, ch2∈{1, . . . , C}, where C is the number of channels in each single embedding, LeftAct(res1, ch1) is a linear operation (e.g., a projection e.g. defined by a matrix multiplication) applied to the channel ch1 of the single embedding indexed by “res1”, and RightAct(res2, ch2) is a linear operation (e.g., a projection e.g. defined by a matrix multiplication) applied to the channel ch2 of the single embedding indexed by “res2”. The result of the outer product mean is generated by flattening and linearly projecting the (ch1, ch2) dimensions of the tensor A. Optionally, the pair embedding update block can perform one or more Layer Normalization operations (e.g., as described with reference to Jimmy Lei Ba et al., “Layer Normalization,” arXiv:1607.06450) as part of computing the outer product mean.

Generally, the updated single embeddings 306 encode information about the amino acids in the amino acid sequence of the protein. By incorporating the information encoded in the updated single embeddings into the current pair embeddings (i.e., by way of the outer product mean 502), the pair embedding update block 500 can enhance the information content of the current pair embeddings.

After updating the current pair embeddings 304 using the updated single embeddings (i.e., by way of the outer product mean 502), the pair embedding update block 308 updates the current pair embeddings in each row of an arrangement of the current pair embeddings into an N×N array using a self-attention operation (i.e., a “row-wise” self-attention operation) that is conditioned on the current pair embeddings. More specifically, the pair embedding update block 500 provides each row of current pair embeddings to a “row-wise” self-attention block 504 that is also conditioned on the current pair embeddings, e.g., as described with reference to FIG. 3, to generate updated pair embeddings for each row. Optionally, the pair embedding update block can add the input to the row-wise self-attention block 504 to the output of the row-wise self-attention block 504.

The pair embedding update block 500 then updates the current pair embeddings in each column of the N×N array of current pair embeddings using a self-attention operation (i.e., a “column-wise” self-attention operation) that is also conditioned on the current pair embeddings. More specifically, the pair embedding update block 500 provides each column of current pair embeddings to a “column-wise” self-attention block 506 that is also conditioned on the current pair embeddings to generate updated pair embeddings for each column. Optionally, the pair embedding update block can add the input to the column-wise self-attention block 506 to the output of the column-wise self-attention block 506.

The pair embedding update block 500 then processes the current pair embeddings using a transition block 508, e.g., that applies one or more fully-connected neural network layers to the current pair embeddings. Optionally, the pair embedding update block 500 can add the input to the transition block 508 to the output of the transition block 508.

The pair embedding update block can output the updated pair embeddings 308 resulting from the operations performed by the row-wise self-attention block 504, the column-wise self-attention block 506, and the transition block 508.

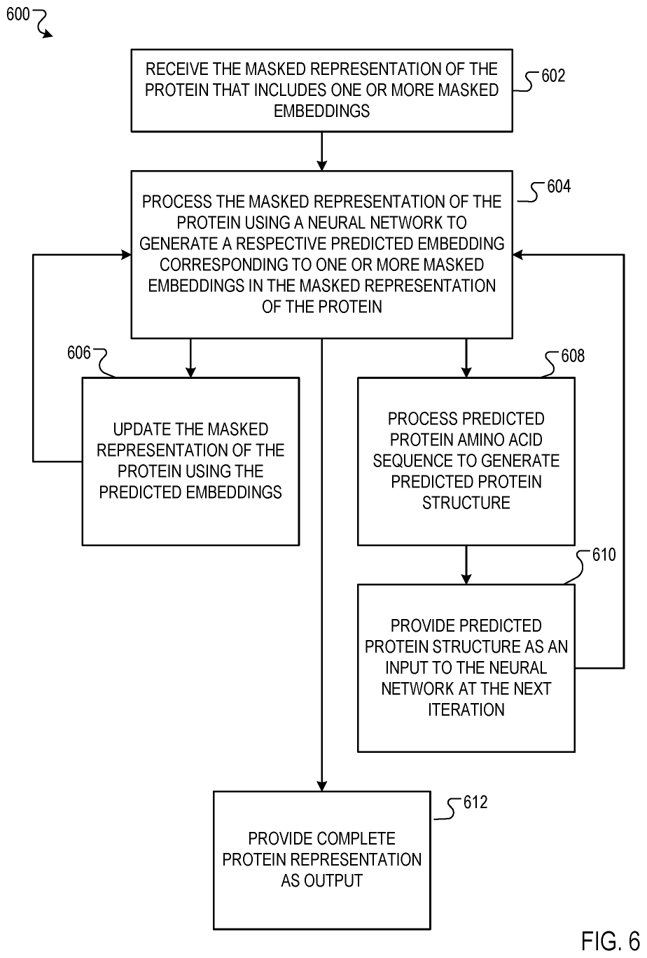

FIG. 6 is a flow diagram of an example process 600 for unmasking a masked representation of a protein using a protein reconstruction neural network. For convenience, the process 600 will be described as being performed by a system of one or more computers located in one or more locations. For example, a protein reconstruction system, e.g., the protein reconstruction system 100 of FIG. 1, appropriately programmed in accordance with this specification, can perform the process 600.

The system receives the masked representation of the protein (602). The masked representation of the protein includes: (i) a representation of an amino acid sequence of the protein that includes a set of embeddings that each correspond to a respective position in the amino sequence of the protein, and (ii) a representation of a structure of the protein that includes a set of embeddings that each correspond to a respective structural feature of the protein. At least one of the embeddings included in the masked representation of the protein is masked.

Steps 604-610, which are described next, can be performed at each of one or more iterations.

The system processes the masked representation of the protein using the protein reconstruction neural network to generate a respective predicted embedding corresponding to one or more masked embeddings that are included in the masked representation of the protein (604). A predicted embedding corresponding to a masked embedding in the representation of the amino acid sequence of the protein defines a prediction for an identity of an amino acid at a corresponding position in the amino acid sequence. A predicted embedding corresponding to a masked embedding in the representation of the structure of the protein defines a prediction for a corresponding structural feature of the protein.

Optionally, if the current iteration is after the first iteration, then the system can provide a predicted protein structure generated at the previous iteration (as will be described in more detail in steps 608-610) as an additional input to the protein reconstruction neural network, i.e., in addition to the masked representation of the protein.

In some implementations, the system can update the masked representation of the protein by replacing a so-called proper subset of the masked embeddings (i.e. a subset not including all the masked embeddings) in the masked representation of the protein by corresponding predicted embeddings (606). The system can then proceed to the next iteration (e.g., by returning to step 604), and at the next iteration, the system can process the updated masked representation of the protein using the protein reconstruction neural network to generate respective predicted embeddings corresponding to one or more remaining masked embeddings that are included in the masked representation of the protein.

In some implementations, the representation of the amino acid sequence of the protein comprises one or more masked embeddings, the system identifies a predicted amino acid sequence of the protein where each masked embedding in the representation of the amino acid sequence is replaced by a corresponding predicted embedding. The system can process the predicted amino acid sequence using a protein folding neural network to generate data defining a predicted protein structure of the predicted amino acid sequence (608). Any protein folding neural network may be used, e.g. based on a published approach or on software such as AlphaFold2 (available open source). The system can then proceed to the next iteration (i.e., by returning to step 604), and at the next iteration, the system can provide the predicted protein structure as an additional input to the protein reconstruction neural network (i.e., in addition to the masked protein representation) (610). The protein reconstruction neural network can then process the predicted protein structure and the masked protein representation to generate new predicted embeddings at the next iteration.

In some implementations, the system can perform both step 606 (i.e., updating the masked representation of the protein using the predicted embeddings) and steps 608-610 (i.e., processing the predicted amino acid sequence to generate a predicted protein structure and providing the predicted protein structure as an additional input to the protein reconstruction neural network, as previously described) at one or more iterations.

The system can determine that the iterative process is complete, e.g., after each masked embedding in the masked protein representation has been replaced by a corresponding predicted embedding. The system can then provide a complete protein representation, i.e., where all the masked embeddings of the masked protein representation have been replaced by corresponding predicted embeddings generated over the course of the sequence of iterations, as an output.

In general the system can be used to determine a predicted structure of a (target) protein, polypeptide ligand, or antibody by generating predicted embeddings that define a complete protein structure representation for the (target) protein, polypeptide ligand, or antibody. This can be achieved e.g. when the masked representation of the protein comprises a complete representation of the amino acid sequence of the (target) protein, polypeptide ligand, or antibody, and the representation of the structure of the protein comprises a fully masked representation of the structure of the (target) protein, polypeptide ligand, or antibody.

Some further applications of the system are described below.

The system may be used to obtain a ligand such as a drug or a ligand of an industrial enzyme. For example a method of obtaining a ligand may comprise obtaining a target amino acid sequence for a target protein, and using the target amino acid sequence to determine the (tertiary) structure of the target protein. The method may involve evaluating an interaction of one or more candidate ligands with the structure of the target protein and selecting one or more of the candidate ligands as the ligand dependent on the result. Evaluating the interaction may comprise evaluating binding of the candidate ligand with the structure of the target protein, e.g. to identify a ligand that binds with sufficient affinity for a biological effect. The candidate ligand may be an enzyme. The evaluating may comprise evaluating an affinity between the candidate ligand and the target protein, or a selectivity of the interaction. The candidate ligand(s) may be derived from a database of candidate ligands, or by modifying ligands in a database of candidate ligands, or by stepwise or iterative assembly or optimization of a candidate ligand. The evaluation may be performed e.g. using a computer-aided approach in which graphical models of the candidate ligand and target protein structure are displayed for user-manipulation, or the evaluation may be performed partially or completely automatically, e.g. using standard protein-ligand docking software. The evaluation may comprise determining an interaction score for the candidate ligand e.g. dependent upon a strength or specificity of the interaction e.g. a score dependent on binding free energy. A candidate ligand may be selected dependent upon its score.

In some implementations the target protein comprises a receptor or enzyme and the ligand is an agonist or antagonist of the receptor or enzyme. In some implementations the method may be used to identify the structure of a cell surface marker. This may then be used to identify a ligand, e.g. an antibody or a label such as a fluorescent label, which binds to the cell surface marker. This may be used to identify and/or treat cancerous cells. In some implementations the candidate ligand(s) may comprise small molecule ligands, e.g. organic compounds with a molecular weight of <900 daltons. In some other implementations the candidate ligand(s) may comprise polypeptide ligands i.e. defined by an amino acid sequence.

Some implementations of the system may be used to determine the structure of a candidate polypeptide ligand, e.g. a drug or a ligand of an industrial enzyme. The interaction of this with a target protein structure may then be evaluated; the target protein structure may have been determined using a computer-implemented method as described herein or using conventional physical investigation techniques such as x-ray crystallography and/or magnetic resonance techniques.

Thus the system may be used to obtain a polypeptide ligand, e.g. the molecule or its sequence. This may comprise obtaining an amino acid sequence of one or more candidate polypeptide ligands and performing a method as described above, using the amino acid sequence of the candidate polypeptide ligand as the sequence of amino acids, to determine a (tertiary) structure of the candidate polypeptide ligand. The structure of a target protein may be obtained e.g. in silico or by physical investigation, and an interaction between the structure of each of the one or more candidate polypeptide ligands and the target protein structure may be evaluated. One of the one or more of the candidate polypeptide ligands may be selected as the polypeptide ligand dependent on a result of the evaluation. As before evaluating the interaction may comprise evaluating binding of the candidate polypeptide ligand with the structure of the target protein e.g. identifying a ligand that binds with sufficient affinity for a biological effect, and/or evaluating an association of the candidate polypeptide ligand with the structure of the target protein which has an effect on a function of the target protein e.g. an enzyme, and/or evaluating an affinity between the candidate polypeptide ligand and the structure of the target protein, or evaluating a selectivity of the interaction. In some implementations the polypeptide ligand may be an aptamer. Again the polypeptide candidate ligand(s) may be selected according to which have the highest affinity.

As before the selected polypeptide ligand may comprise a receptor or enzyme and the ligand may be an agonist or antagonist of the receptor or enzyme. In some implementations the polypeptide ligand may comprises an antibody and the target protein comprises an antibody target i.e. an antigen, for example a virus, in particular a virus coat protein, or a protein expressed on a cancer cell. In these implementations the antibody binds to the antigen to provide a therapeutic effect. For example, the antibody may bind to the antigen and act as an agonist for a particular receptor; alternatively, the antibody may prevent binding of another ligand to the target, and hence prevent activation of a relevant biological pathway.

Such methods may include synthesizing i.e. making the small molecule or polypeptide ligand. The ligand may be synthesized by any conventional chemical techniques and/or may already be available e.g. may be from a compound library or may have been synthesized using combinatorial chemistry.

The method may further comprise testing the ligand for biological activity in vitro and/or in vivo. For example the ligand may be tested for ADME (absorption, distribution, metabolism, excretion) and/or toxicological properties, to screen out unsuitable ligands. The testing may comprise e.g. bringing the candidate small molecule or polypeptide ligand into contact with the target protein and measuring a change in expression or activity of the protein.

In some implementations a candidate (polypeptide) ligand may comprise: an isolated antibody, a fragment of an isolated antibody, a single variable domain antibody, a bi- or multi-specific antibody, a multivalent antibody, a dual variable domain antibody, an immuno-conjugate, a fibronectin molecule, an adnectin, an DARPin, an avimer, an affibody, an anticalin, an affilin, a protein epitope mimetic or combinations thereof. A candidate (polypeptide) ligand may comprise an antibody with a mutated or chemically modified amino acid Fc region, e.g. which prevents or decreases ADCC (antibody-dependent cellular cytotoxicity) activity and/or increases half-life when compared with a wild type Fc region.

Misfolded proteins are associated with a number of diseases. The system can be used for identifying the presence of a protein mis-folding disease. This may comprise obtaining an amino acid sequence of a protein and performing a method as described above using the amino acid sequence of the protein to determine a structure of the protein, obtaining a structure of a version of the protein obtained from a human or animal body e.g. by conventional (physical) methods, and then comparing the structure of the protein with the structure of the version obtained from the body, identifying the presence of a protein mis-folding disease dependent upon the result. That is, mis-folding of the version of the protein from the body may be determined by comparison with the determined structure. In general identifying the presence of a protein mis-folding disease may involve obtaining an amino acid sequence of a protein, using an amino acid sequence of the protein to determine a structure of the protein, as described herein, and comparing the structure of the protein with the structure of a baseline version of the protein, identifying the presence of a protein mis-folding disease dependent upon a result of the comparison. For example the compared structures may be those of a mutant and wild-type protein. In implementations the wild-type protein may be used as the baseline version but in principle either may be used as the baseline version.

In some implementations the system can be used to identify active/binding/blocking sites on a target protein from its amino acid sequence.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more data processing apparatus for unmasking a masked representation of a protein using a protein reconstruction neural network, the method comprising:
receiving the masked representation of the protein,
wherein the masked representation of the protein comprises: (i) a representation of an amino acid sequence of the protein that comprises a plurality of embeddings that each correspond to a respective position in the amino sequence of the protein, and (ii) a representation of a structure of the protein that comprises a plurality of embeddings that each correspond to a respective structural feature of the protein,
wherein at least one of the embeddings included in the masked representation of the protein is masked; and
processing the masked representation of the protein using the protein reconstruction neural network to generate a respective predicted embedding corresponding to one or more masked embeddings that are included in the masked representation of the protein,
wherein a predicted embedding corresponding to a masked embedding in the representation of the amino acid sequence of the protein defines a prediction for an identity of an amino acid at a corresponding position in the amino acid sequence,
wherein a predicted embedding corresponding to a masked embedding in the representation of the structure of the protein defines a prediction for a corresponding structural feature of the protein.

2. The method of claim 1, further comprising:
updating the masked representation of the protein by replacing a proper subset of the masked embeddings in the masked representation of the protein by corresponding predicted embeddings;
processing the updated masked representation of the protein using the protein reconstruction neural network to generate respective predicted embeddings corresponding to one or more remaining masked embeddings that are included in the masked representation of the protein.

3. The method of claim 1, wherein the representation of the amino acid sequence of the protein comprises one or more masked embeddings, and further comprising:
processing a predicted amino acid sequence of the protein, defined by replacing each masked embedding in the representation of the amino acid sequence by a corresponding predicted embedding, using a protein folding neural network to generate data defining a predicted protein structure of the predicted amino acid sequence; and
processing both: (i) the masked representation of the protein, and (ii) the predicted protein structure of the predicted amino acid sequence, using the protein reconstruction neural network to generate a new predicted embedding corresponding to one or more masked embeddings that are included in the masked representation of the protein.

4. The method of claim 1, wherein each masked embedding included in the masked representation of the protein is a default embedding.

5. The method of claim 4, wherein the default embedding comprises a vector of zeros.

6. The method of claim 1, wherein each predicted embedding corresponding to a masked embedding in the representation of the structure of the protein defines a prediction for a spatial distance between a corresponding pair of amino acids in the structure of the protein.

7. The method of claim 1, wherein at least one of the embeddings of the representation of the amino acid sequence of the protein is masked.

8. The method of claim 1, wherein at least one of the embeddings of the representation of the structure of the protein is masked.

9. The method of claim 1, wherein the representation of the amino acid sequence of the protein comprises a plurality of single embeddings that each correspond to a respective position in the amino acid sequence of the protein;
wherein the representation of the structure of the protein comprises a plurality of pair embeddings that each corresponding to a respective pair of positions in the amino acid sequence of the protein;
wherein the protein reconstruction neural network comprises a sequence of update blocks;
wherein each update block has a respective set of update block parameters and performs operations comprising:
receiving current pair embeddings and current single embeddings;
updating the current single embeddings, in accordance with values of the update block parameters of the update block, based on the current pair embeddings; and
updating the current pair embeddings, in accordance with the values of the update block parameters of the update block, based on the updated single embeddings; and
wherein a final update block in the sequence of update blocks generates final pair embeddings and final single embeddings.

10. The method of claim 9, wherein the protein reconstruction neural network performs further operations comprising, for each of one or more masked single embeddings in the representation of the amino acid sequence of the protein:
generating the predicted embedding for the masked single embedding based on the corresponding final single embedding generated by the final update block.

11. The method of claim 9, wherein the protein reconstruction neural network performs further operations comprising, for each of one or more masked pair embeddings in the representation of the amino acid sequence of the protein:
generating the predicted embedding for the masked pair embedding based on the corresponding final pair embedding generated by the final update block.

12. The method of claim 9, wherein updating the current single embeddings based on the current pair embeddings comprises:
updating the current single embeddings using attention over the current single embeddings, wherein the attention is conditioned on the current pair embeddings.

13. The method of claim 12, wherein updating the current single embeddings using attention over the current single embeddings comprises:
generating, based on the current single embeddings, a plurality of attention weights;
generating, based on the current pair embeddings, a respective attention bias corresponding to each of the attention weights;
generating a plurality of biased attention weights based on the attention weights and the attention biases; and
updating the current single embeddings using attention over the current single embeddings based on the biased attention weights.

14. The method of claim 9, wherein updating the current pair embeddings based on the updated single embeddings comprises:
applying a transformation operation to the updated single embeddings; and
updating the current pair embeddings by adding a result of the transformation operation to the current pair embeddings.

15. The method of claim 14, wherein the transformation operation comprises an outer product operation.

16. The method of claim 14, wherein updating the current pair embeddings based on the updated single embeddings further comprises, after adding the result of the transformation operation to the current pair embeddings:
updating the current pair embeddings using attention over the current pair embeddings, wherein the attention is conditioned on the current pair embeddings.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A system comprising:
one or more computers; and
one or more storage devices communicatively coupled to the one or more computers, wherein the one or more storage devices store instructions that, when executed by the one or more computers, cause the one or more computers to perform operations for unmasking a masked representation of a protein using a protein reconstruction neural network, the operations comprising:
receiving the masked representation of the protein,
wherein the masked representation of the protein comprises: (i) a representation of an amino acid sequence of the protein that comprises a plurality of embeddings that each correspond to a respective position in the amino sequence of the protein, and (ii) a representation of a structure of the protein that comprises a plurality of embeddings that each correspond to a respective structural feature of the protein,
wherein at least one of the embeddings included in the masked representation of the protein is masked; and
processing the masked representation of the protein using the protein reconstruction neural network to generate a respective predicted embedding corresponding to one or more masked embeddings that are included in the masked representation of the protein,
wherein a predicted embedding corresponding to a masked embedding in the representation of the amino acid sequence of the protein defines a prediction for an identity of an amino acid at a corresponding position in the amino acid sequence,
wherein a predicted embedding corresponding to a masked embedding in the representation of the structure of the protein defines a prediction for a corresponding structural feature of the protein.

30. One or more non-transitory computer storage media storing instructions that when executed by one or more computers cause the one or more computers to perform operations for unmasking a masked representation of a protein using a protein reconstruction neural network, the operations comprising:
receiving the masked representation of the protein,
wherein the masked representation of the protein comprises: (i) a representation of an amino acid sequence of the protein that comprises a plurality of embeddings that each correspond to a respective position in the amino sequence of the protein, and (ii) a representation of a structure of the protein that comprises a plurality of embeddings that each correspond to a respective structural feature of the protein,
wherein at least one of the embeddings included in the masked representation of the protein is masked; and
processing the masked representation of the protein using the protein reconstruction neural network to generate a respective predicted embedding corresponding to one or more masked embeddings that are included in the masked representation of the protein,
wherein a predicted embedding corresponding to a masked embedding in the representation of the amino acid sequence of the protein defines a prediction for an identity of an amino acid at a corresponding position in the amino acid sequence,
wherein a predicted embedding corresponding to a masked embedding in the representation of the structure of the protein defines a prediction for a corresponding structural feature of the protein.
